(19) United States
(12) Patent Application Publication
(10) Pub. No.: US 2019/0333632 A1
(43) Pub. Date: Oct. 31, 2019

(54) DEVICE MANAGEMENT SYSTEM

(71) Applicant: SMARTLINE HOLDINGS PTY LTD, Palmwoods (AU)

(72) Inventor: William Hugh Dawkins Smart, Queensland (AU)

(21) Appl. No.: 16/508,822

(22) Filed: Jul. 11, 2019

Related U.S. Application Data
(63) Continuation of application No. 15/117,376, filed on Aug. 8, 2016.

(30) Foreign Application Priority Data
Feb. 7, 2014 (AU) 2014900373

(51) Int. Cl. G16H 40/20; G06Q 10/00; G06Q 50/22; A61B 1/12; G16H 40/40; G06Q 10/10

(52) U.S. Cl. CPC G16H 40/20; G06Q 10/00; G06Q 50/22; A61B 2090/701; G16H 40/40; G06Q 10/109; A61L 2202/14; A61B 1/123

(57) ABSTRACT

A device management system, the system including: a tag associated with a device; a cleaning module adapted to receive a validity indication that the device has been cleaned; a status device adapted to determine at least one location of the device via its proximity to the tag; and wherein a re-cleaning time of the device is determined according to the at least one location of the device and the validity indication that the device has been cleaned.

### CROSS-REFERENCE TO RELATED APPLICATION(S)

The present application is a continuation of U.S. application Ser. No. 15/117,376 filed on Aug. 8, 2016, which in turn is the national phase of PCT Application No. PCT/AU2015/050042 filed on Feb. 6, 2015, which in turn claims priority to Australian Application No. 2014900373 filed on Feb. 7, 2014.

### FIELD OF THE INVENTION

The invention relates to a device management system. In particular, the invention relates, but is not limited, to a device management system for determining a re-cleaning time of a flexible scope, such as an endoscope, and validating the cleaning and storage times of the scope.

### BACKGROUND TO THE INVENTION

Reference to background art herein is not to be construed as an admission that such art constitutes common general knowledge in Australia or elsewhere.

Medical devices are associated with a definitive risk of bacterial and fungal infections. To reduce the incidences of infection, guidelines have been developed. For example, in most countries throughout the world, flexible scopes, such as endoscopes, must be cleaned after a period of storage. Typically, if a scope has been washed with alcohol, it can be stored for up to 72 hours in a suitable clean storage cabinet. If no alcohol was used, it may only be stored up to 10 hours.

Currently, to track how long a scope has been in storage, a timer is typically set on the front of the clean storage cabinet. Whilst this gives a general idea on the amount of time the flexible scope has been in the storage cabinet, there are a number of shortfalls in this system. For example, a user may incorrectly assume that the scope has been washed in alcohol and set the storage time as 72 hours.

Furthermore, nothing in these current systems tracks the amount of time the flexible scope is out of the clean storage cabinet. Leaving flexible scopes out of the clean storage cabinet accelerates bacterial and fungal growth. Therefore, these scopes should be stored for a shorter period of time before they are re-washed. Similarly, changes in the storage cabinet environment may reduce the time the scopes should be stored. However, current systems are not adapted to track and indicate how long a flexible scope should be stored in a particular environment and whether the scope should be re-washed.

### OBJECT OF THE INVENTION

It is an aim of this invention to provide a device management system which overcomes or ameliorates one or more of the disadvantages or problems described above, or which at least provides a useful alternative.

Other preferred objects of the present invention will become apparent from the following description.

### SUMMARY OF INVENTION

In one form, although not necessarily the only or broadest form, the invention resides in a device management system, the system including:

a tag associated with a device;

a cleaning module adapted to receive a validity indication that the device has been cleaned;

a status device adapted to determine at least one location of the device via its proximity to the tag; and

wherein a re-cleaning time of the device is determined according to the at least one location of the device and the validity indication that the device has been cleaned.

Preferably, the tag is a radio frequency identification (RFID) tag. Typically, the RFID tag is active and includes its own power source. Alternatively, the RFID tag is passive and requires a power signal to operate. Preferably, the device is in the form of medical equipment. Preferably, the device is a scope. Preferably, the device is flexible scope or a rigid scope. Preferably, the device is an endoscope, gastroscope, bronchoscope, duodenoscope, enterscope, ultrasound scope, toe probe, truss probe, Brachy probe and/or ENT flexible or rigid scope. Typically, the scope is in the form of an endoscope.

Preferably, the device management system includes at least one process module. Preferably, the at least one process module is configured to monitor workflow associated with the device. Preferably, the at least one process module allows an operator to register whether at least one process has been performed. Preferably, the at least one process is performed prior to the cleaning module receiving the validity indication. Preferably, the at least one process includes whether the device has been washed, brushed, leak tested, rinsed and/or manually inspected.

Preferably, the at least one process module is configured to register an associated condition of the device depending on an operator input. Preferably, the operator input is associated with the tag. For example, the tag may be located proximate to the at least one process module for a first predetermined time to indicate a first condition. A time greater than the first predetermined time may indicate a second condition. Preferably, the associated condition of the device provides a related procedure of the device. Preferably, the related procedure includes cleaning the device within a predetermined time.

Preferably, the device management system includes a plurality of process modules. Preferably, the plurality of process modules assist in making an operator follow a sequence of processes. Preferably, in response to the operator missing a process in the sequence of processes, an indication is given to the operator by at least one of the plurality of process modules to return to a previous process.

Preferably, the device management system includes a system monitor. Typically, the system monitor is in communication with the cleaning module, the status device, the tag and/or the at least one process module. Normally, the system monitor receives information from the cleaning module, status device, the tag and/or the at least one process module allowing the system monitor to monitor the current status of the device. Furthermore, the system monitor is adapted to provide a single activity log from activities recorded by the at least one process module, the cleaning module and/or the status device.

Preferably, the cleaning module is adapted to record information on the tag regarding when the device was cleaned and/or the cleaning performed on the device. Even more preferably, the cleaning module records an initial time on the tag when the device should be re-cleaned. Alternatively, or additionally, the cleaning module may communicate with the status device and/or the system monitor regarding when the device was cleaned, the cleaning performed on the device and/or when the device should be re-cleaned. Ideally, the cleaning module keeps a log of the activities performed at the cleaning module. Preferably, the device is cleaned by an associated cleaning unit. Preferably, the cleaning module is configured to receive the validity indication that the device has been cleaned from the associated cleaning unit.

Normally, the cleaning module includes a display. The display of the cleaning module usually indicates a current status of cleaning or the required information needed to perform cleaning.

Typically, an operator has to be identified as being on an authorised list by the cleaning module to undertake cleaning. Typically, authorised cleaning operators are identified by an operator tag. Preferably, the operator tag is an active RFID tag.

Normally, the authorised list of cleaning operators is stored on the cleaning module. Alternatively, the authorised list of cleaning operators is stored on the system monitor. Authorised cleaning operators are usually added to the list of authorised cleaning operators by reading their operator tag after authorising their operator tag to be added to the authorised list. To authorise a cleaning operator to be added to the list, typically a separate operator tag with authorisation is read by the cleaning module.

Preferably, the status device is adapted to communicate with the tag. Preferably, the cleaning module is configured to read information from the tag regarding the sequence of processes respectively performed at the process modules. Preferably, the cleaning module keeps a log of information received from the tag. Typically, the status device is in the at least one location in the form of a clean storage environment. Preferably, the clean storage environment is a clean storage cabinet.

Normally, the status device establishes communication with the tag when the tag is proximate and the device is in the clean storage environment. Typically, the status device reads information from the tag regarding the validity indication that the device has been cleaned. In particular, the status device usually reads information regarding when the device was cleaned and the cleaning performed on the device. Alternatively, or additionally, the status device reads information regarding the initial time when the device should be re-cleaned.

Preferably, the status device determines the re-cleaning time of the device according to the at least one location of the device and the validity indication that the device has been cleaned. For example, the status device establishes from when the device was cleaned and its time out of the clean storage environment when the device should be re-cleaned.

Alternatively, the status device may receive communication from the system monitor which determines the re-cleaning time of the device according to the at least one location of the device and the validity indication that the device has been cleaned. Normally, the status device indicates when the device should be re-cleaned via a display. Preferably, the status device is configured to determine cross-contamination. Preferably, the status device is configured to determine cross-contamination by determining whether the device has been processed correctly through the at least one process module and/or the validity indication that the device has been cleaned was received.

Typically, when the device is removed from the clean storage environment, the status device records when the device should be re-cleaned on the tag. This information may also be communicated to the system monitor. Preferably, the status device maintains log data on information received and transferred to the tag associated with the device. Normally, the display of the status device is located on a rotary storage disc in the clean storage cabinet.

Preferably, the device management system includes a storage controller. Typically, the storage controller monitors the conditions of the clean storage environment. That is, the storage controller monitors the conditions of the clean storage environment in the form of the clean storage cabinet. Ideally, depending on the conditions in the clean storage environment, the storage controller sends information to adjust the time until the device should be re-cleaned. For example, the storage controller may send information to reduce the time until the device should be re-cleaned if the temperature in the clean storage environment is too high.

Typically, the storage controller is in communication with the status device. Alternatively, the storage controller is in communication with the system monitor and/or the tag. The storage controller may control devices in the clean storage environment to maintain the clean storage environment within predefined parameters.

Typically, the device management system includes a master station. The master station is adapted to program the tag and operator tag. Preferably, the master station is a stand alone unit. Alternatively, the master station is adapted to communicate with other components such as the system monitor. The master station may lock the information programmed onto the tag and operator tag if required. The master station may also keep log data of the programming of the tag and operator tag. This log data may be communicated to the system monitor.

Normally, the device management system also includes a receipt printer. The receipt printer is adapted to receive and print information from any one of the tag, operator tag, cleaning module, status device, storage controller and system monitor. Typically, when specific events are recorded at the cleaning module or at the status display, a message is sent to the receipt printer for printing. For example, the receipt printer will print when the device was cleaned, when the device was stored into the clean storage environment, when the device was removed from clean storage environment and when the scope should be re-cleaned. Alternatively, or additionally, the receipt printer may read information from the tag and print information relating to the activities of the device.

In another form, the invention resides in a method of determining a cleaning cycle of a device, the method including the steps of:

receiving an indication that the device has been cleaned;

determining at least one location of the device via a tag associated with the device, subsequent to receiving the indication that the device has been cleaned; and

determining a re-cleaning time of the device according to the at least one location and the indication that the device has been cleaned.

Preferably, before receiving an indication that the device has been cleaned, the method further includes loading the device in a cleaning unit. Preferably, the step of receiving the indication that the device has been cleaned includes receiving an indication from a cleaning unit though a cleaning module.

Preferably, before receiving an indication that the device has been cleaned, the method further includes the step of registering whether at least one process has been performed with the device. Preferably, the step of registering whether the at least one process has been performed with the device includes registering the tag with a process module. Preferably, the step of registering whether the at least one process has been performed with the device includes registering an operator tag with the process module to determine who performed the at least one process. Preferably, the at least one process includes washing, wiping, brushing, leak testing, rinsing and/or manually inspecting the device.

Preferably, the step of registering whether at least one process has been performed with the device includes registering an associated condition of the device. Preferably, the step of registering the associated condition of the device includes presenting the tag associated with the device to the process module for a predetermined time. Preferably, the associated condition of the device provides a related procedure of the device. Preferably, the related procedure includes cleaning the device within a predetermined time.

Preferably, the step of receiving an indication that the device has been cleaned includes recording when the device was cleaned. Even more preferably, the step of receiving an indication that the device has been cleaned includes recording the type of cleaning for the device. Normally, the step of receiving an indication that the device has been cleaned also includes identifying and recording the operator who cleaned the device.

Normally, before receiving an indication that the device has been cleaned, the method further includes the step of adding an operator to an authorised list. Typically, the step of adding an operator to the authorised list includes identifying an authorised operator followed by identifying an operator tag.

Preferably, the step of determining the at least one location of the device via the tag includes determining whether the device is in a clean storage environment. Typically, the step of determining the at least one location of the device via the tag includes bringing the tag into proximity with a status device. Preferably, when the tag is brought into proximity with the status device, the device is in the clean storage environment.

Preferably, the step of determining the re-cleaning time of the device includes adjusting the re-cleaning time of the device depending on whether the device is in or out of the clean storage environment. Normally, the step of determining the re-cleaning time of the device also includes adjusting the re-cleaning time of the device depending on the type of cleaning performed on the device.

Usually, the method of determining the cleaning cycle of the device includes monitoring parameters in the clean storage environment. Preferably, determining the re-cleaning time of the device may be adjusted according to the monitored parameters in the clean storage environment. Preferably, the method of determining the cleaning cycle of the device includes determining whether the device has been cross-contaminated from one or more other devices. Preferably, the step of determining whether the device has been cross-contaminated includes determining whether the other device is not clean.

Preferably, the method of determining the cleaning cycle of the device also includes printing a receipt. Preferably, the receipt contains information such as when the scope should be re-cleaned.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 illustrates a schematic of a device management system according to an embodiment of the invention;

FIG. 2 illustrates a flow diagram for determining a cleaning cycle of the device; and

FIG. 3 illustrates a flow diagram illustrating part of a process path in FIG. 2.

### DETAILED DESCRIPTION OF THE DRAWINGS

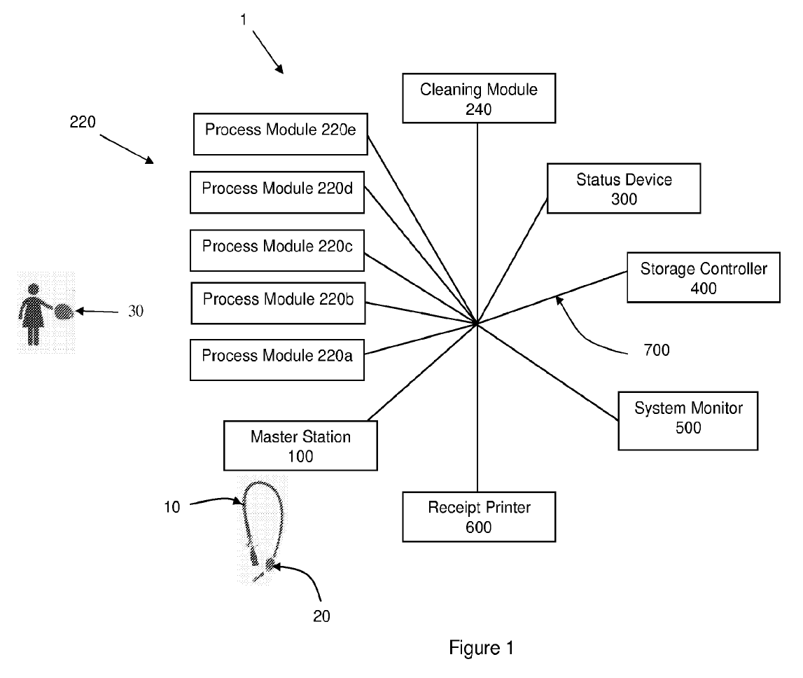

FIG. 1 illustrates a device management system 1 according to an embodiment of the invention. The device management system 1 includes a device in the form of a scope 10, a tag 20, an operator tag 30, a master station 100, process modules 220, a cleaning module 240, a status device 300, a storage controller 400, a system monitor 500, a receipt printer 600 and a network 700.

The tag 20 is attached to the scope 10. The tag 20 and operator tag 30 are adapted to store and process information along with transferring and receiving information. In this embodiment, the tag 20 and operator tag 30 are in the form of radio frequency identification (RFID) tags. The tag 20 and operator tag 30 are active RFID tags and include their own battery power. However, it would be appreciated by a person skilled in the art that the tag 20 and operator tag 30 may be passive and require a power signal from the reader device to operate.

In this embodiment, the master station 100 operates as a stand-alone device. However, the master station 100 includes a communication interface to communicate with other components such as the process modules 220, the cleaning module 240, status device 300, storage controller 400, system monitor 500 and receipt printer 600, in the device management system 1, via the network 700. The communication interface of the master station 100 may also communicate with other components external to the device management system 1.

The master station 100 programs the tag 20 and operator tag 30. In particular, the master station 100 programs the tag 20 with information including i) a Unique Identification Number (UID); ii) the type of scope 10; iii) the name of the scope 10; iv) the amount of time the scope 10 is allowed outside of a clean storage environment once it has been cleaned; and v) the length of time the scope 10 can be stored in the clean storage environment. Moreover, the master station 100 may program a particular process or clean path for the scope 10. For example, the scope 10 may be required to proceed through the process modules 220 and cleaning module 240, as outlined below, or may proceed directly to the cleaning module 240.

Further to the above, data entered into the master station 100 and/or onto the tag 20, may also allow the identity of the scope 10 to be established. For example, if a particular barcode of the scope 10 is entered as data, along with the associated brand name of the scope 10, the type of scope 10 may be established.

The master station 100 also programs the operator tag 30 to include information such as the name of the operator and whether they are a washer operator or a washer authoriser, as further discussed below. The master station 100 may also be used to program other special tags as required by the device management system 1.

Once the tag 20 and operator tag 30 have been programmed, they can be used with the other components in the device management system 1. If required, the master station 100 may lock the information on the tag 20 and operator tag 30 so that this information cannot be altered. Furthermore, the master station keeps log data on the programming of the tag 20 and operator tag 30 for future reference and auditing.

The process modules 220 monitor operator workflow associated with the scope 10 before proceeding to an automatic cleaning unit associated with the cleaning module 240. That is, the process modules 220 allow the operator to register whether certain processes have been performed before proceeding to the cleaning unit or elsewhere. As outlined below, five separate processes are undertaken in this embodiment before proceeding to the cleaning unit. However, as would be appreciated by a person skilled in the art, further processes may be added as required or dictated by guidelines or regulations.

The process modules 220 in this embodiment are configured to register associated conditions of the scope 10 depending on an operator input. That is, the operator may register a short swipe or a long swipe with the process modules 220. The short swipe may, for instance, involve holding the tag 20 proximate to the process module 220 for less than 4 second. The long swipe may, for instance, involve holding the tag 20 proximate to the process module 220 for greater than 4 second. The long swipe may indicate that the scope 10 is heavily soiled and requires to be cleaned within a predetermined time.

Further to the above, five process modules 220 in the form of process modules 220a, 220b, 220c, 220d, 220e are located at five work stations in this embodiment. The first process module 220a is associated with an operating theatre. The first process module 220a allows the operator to register that the scope 10 has been washed/wiped with detergent after being used on a patient. The process module 220a is configured to record on the tag 20 that the scope 10 has been washed/wiped after the operator swipes their operator tag 30 proximate to the first process module 220a followed by the tag 20. This process also allows the identity of the operator to be recorded on the tag 20.

Furthermore, depending on the duration of the swipe of the tag 20 (i.e. a short swipe or long swipe), an associated condition of the scope 10 is registered on the tag 20. For example, if a long swipe is registered (i.e. greater than four seconds), the scope is identified as being heavily soiled and requires cleaning to be completed within one hour.

The second process module 220b is associated with a first sink station. The second process module 220b allows the operator to register that channels (i.e. tubes) within the scope 10 have been brushed individually (according to guidelines). To this end, it is noted that if the washing/wiping of the scope 10 has not been registered on the tag 20 by the process module 220a, when registering that the channels within the scope 10 have been brushed individually, a display on the process module 220b will direct the operator back to wash/swipe the scope 10 at the previous stage.

Accordingly, the process modules 220 assist in preventing operators missing processes in cleaning/maintaining the scope 10. Furthermore, as would be appreciated, the process modules 220 may assist in making operators follow a particular sequence of processes with the scope 10.

The process module 220b is configured to record on the tag 20 that the channels of the scope 10 have been brushed individually after the operator swipes their operator tag 30 proximate to the second process module 220b followed by the tag 20. This process also allows the identity of the operator to be recorded on the tag 20.

Furthermore, similar to the above, the operator may register an associated condition of the scope 10 depending on an operator input. For example, the operator may swipe (i.e. leave the tag 20 proximate to the process module 220b) for more than four seconds to indicate that the scope 10 requires an alternative treatment than that provided at process module 220c below.

The third process module 220c is associated a second sink station. The third process module 220c allows the operator to register that the scope 10 has been manually washed and rinsed (according to guidelines). Similar to the above, it is noted that if the washing/wiping of the scope 10 and/or brushing of the channels in the scope 10 has not been registered on the tag 20, a display on the process module 220c will direct the operator back to the previous stage(s).

The process module 220c is configured to record on the tag 20 that the scope 10 has been manually washed and rinsed after the operator swipes their operator tag 30 proximate to the third process module 220c followed by the tag 20. This process also allows the identity of the operator to be recorded on the tag 20.

Furthermore, similar to the above, the operator may register an associated condition of the scope 10 depending on an operator input. For example, the operator may swipe (i.e. leave the tag 20 proximate to the process module 220c) for more than four seconds to indicate that the scope 10 requires an alternative treatment than that provided at process module 220d below.

The fourth process module 220d is associated with a leak testing apparatus. The third process module 220d allows the operator to register that the scope 10 has been leak tested (according to guidelines). Similar to the above, if one of the stages at process modules 220a, 220b, 220c has been missed, the fourth process module 220d will redirect the operator to the required process or the required sequence of processes.

The process module 220d is configured to record on the tag 20 that the scope 10 has been leak tested after the operator swipes their operator tag 30 proximate to the fourth process module 220d followed by the tag 20. This process also allows the identity of the operator to be recorded on the tag 20.

Furthermore, similar to the above, the operator may register an associated condition of the scope 10 depending on an operator input. For example, the operator may swipe (i.e. leave the tag 20 proximate to the process module 220d) for more than four seconds to indicate that the scope 10 requires an alternative treatment. That is, for instance, if the scope 10 fails the leak test its seals may have to be replaced.

The fifth process module 220e is associated with a manual inspection. The fifth process module 220e allows the operator to register that the scope 10 has been manually inspected (according to guidelines). Similar to the above, if one of the stages at process modules 220a, 220b, 220c, 220d has been missed, the fifth process module 220e will redirect the operator to the required process or the required sequence of processes.

The process module 220e is configured to record on the tag 20 that the scope 10 has been manually inspected after the operator swipes their operator tag 30 proximate to the fifth process module 220e followed by the tag 20. This process also allows the identity of the operator to be recorded on the tag 20.

Furthermore, similar to the above, the operator may register an associated condition of the scope 10 depending on an operator input. For example, the operator may swipe (i.e. leave the tag 20 proximate to the process module 220e) for more than four seconds to indicate that the scope 10 requires an alternative treatment.

The cleaning module 240 records the cleaning of the scope 10 in an associated cleaning unit. For example, the cleaning module 240 records whether a short, standard or long wash has been used to clean the scope 10 in the associated cleaning unit. Furthermore, other parameters such as whether alcohol was used in the clean cycle are recorded. In this regard, the cleaning module 240 is configured to receive a validity indication from the associated cleaning unit that the scope 10 has been cleaned according to a particular wash cycle.

The cleaning module 240 is adapted to receive and transfer information to the tag 20 and operator tag 30. In this regard, the cleaning module 240 is similar to the process modules 220. The cleaning module 240 keeps a log file of the data received and transferred to and from the tag 20 and operator tag 30. This log file may include the tag 20 UID, the number of cleaning cycles the scope 10 has been through, the sequence of processes performed at the process modules 220, the name of the operator who cleaned the scope 10, time and date. Furthermore, cleaning module 240 has a display to view the data received and transferred to and from the tag 20 and operator tag 30. The cleaning module 240 is also adapted to communicate with other components in the device management system 1, such as the status device 300, system monitor 500 and receipt printer 600, via the network 700. This communication interface may also communicate with other components external to the device management system 1. However, as would be appreciated by a person skilled in the art, the cleaning module 240 may be a standalone unit that communicates primarily with, for example, the tag 20 or operator tag 30.

The status device 300 is adapted to read information stored on the tag 20 and send information to the tag 20. The status device 300 is in communication with other components in the device management system 1, such as the process modules 220, the cleaning module 240, storage controller 400, system monitor 500 and receipt printer 600, via the network 700. Again however, as would be appreciated by a person skilled in the art, the status device 300 may be a standalone unit that communicates primarily with, for example, the tag 20 or operator tag 30.

The status device 300 determines the amount of time remaining until the scope 10 must be re-cleaned. The status device 300 displays the amount of time remaining until the scope 10 must be re-cleaned via a LED display. The status device 300 maintains log data on information received from the tag 20 and the other components in the device management system 1. The status device 300 is located on a rotary disc in the clean storage environment in the form of a clean storage cabinet (not shown) where the scope 10 is stored. The rotary disc is adapted to store a number of scopes 10 about a central pole in the clean storage cabinet and each scope 10 is associated with one status device 300.

The status device 300 is also configured to assist in determining cross-contamination. That is, if the status device 300 detects, via the tag 20, that the scope 10 has not been processed correctly through the process modules 220 or a validity indication has not been received from the cleaning unit and/or the cleaning module 240, the status device 300 is configured to indicate that other scopes adjacent thereto have been contaminated. For example, the LED displays may indicate that the scope 10 and the scopes adjacent thereto must be re-cleaned.

The storage controller 400 monitors and controls conditions in the clean storage cabinet. The storage controller 400 is in communication with the other components in the device management system 1, such as the status device 300 and system monitor 500, via the network 700. Furthermore, the storage controller 400 maintains a log of the conditions in the clean storage cabinet. In particular, the storage controller 400 monitors parameters such as air quality, temperature, humidity, whether the door of the cabinet is open and the flow of clean air through the scope 10 in the clean storage cabinet. The storage controller 400 is adapted to alert a user when the clean storage cabinet is not within a predefined parameter. For example, if the air quality is determined to be outside an acceptable range, the storage controller 400 may indicate that a HPEA filter in the storage cabinet needs replacing. Alternatively, or additionally, the storage controller 400 may control devices in the clean storage cabinet to maintain the cabinet within predefined parameters.

The storage monitor 400 is also adapted to control a lock to a door that is part of the clean storage cabinet. To activate and open the lock, the operator tag 30 needs to be registered at storage controller 400. This allows a record of who accessed the cabinet to be recorded. Furthermore, the lock can be programmed to interlock the door(s) to a room on a ‘pass through’ version of the cabinet. This stops the cabinet door being open at the same time as the doors to the room and, therefore, prevents direct airflow from one room to another via the clean storage cabinet.

The system monitor 500 is used to provide a single view point of the components in the device management system 1. In this regard, the system monitor 500 is able to monitor and collate the log data from the other components in the device management system 1. In particular, the system monitor 500 can query the current status of the scope 10, typically via the status device 300, and provide a summary of the recent activity at the process modules 220 and cleaning module 240. It will be appreciated by a person skilled in the art that the system monitor 500 may determine the re-cleaning time of the scope 10 from information received from the other components in the device management system 1.

The receipt printer 600 is adapted to receive and print information from the master station 100, process modules 220, cleaning module 240, status device 300, storage controller 400 and system monitor 500 via the network 700. However, the receipt printer 600 may also directly receive and print information from the tag 20 and operator tag 30 through a reader on the receipt printer 600.

The network 700 in this embodiment is a RS485 network. However, it would be appreciated by a person skilled in the art that network 700 may take a variety of other forms including wireless. The network 700 may include a data box to monitor and/or manage data on the network 700. The data box may include a display that allows an operator to view how many devices (i.e. scopes 10) are on the network 700. Furthermore. the data box may be configured to manage data on the network 700 in order to reduce the amount of traffic on the network 700. For example. if a tag 20 is in intermittent communication with the status device 300, due to its location. a number of data parcels will be created indicating whether the scope 10 is in or out of the clean storage environment. The data box may moderate these data parcels (i.e. provide a lower sampling rate) to reduce the amount of traffic on the network 700. Moreover, the data box may buffer the data on the network 700 if, for instance, the system monitor 500 has been switched off.

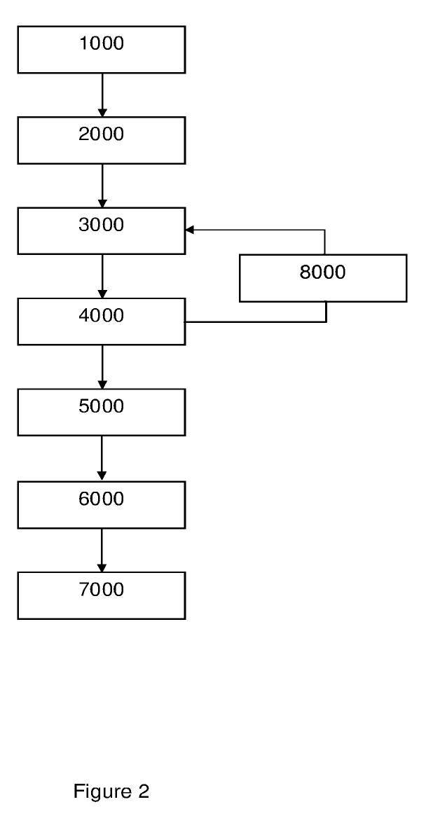

FIG. 2 illustrates a flow diagram of determining a cleaning cycle of the scope 10 (i.e. when the scope 10 should be re-cleaned) according to an aspect of the invention with reference to FIG. 1.

At step 1000, before processing or cleaning the scope 10, an operator must be registered to perform such operations. To do this, an authoriser must swipe their operator tag 30 such that the master station 100 reads the operator tag 30. Following this, the operator presents their own operator tag 30 to the master station 100, which will then add the operator to an authorised list. It would be appreciated that the authoriser may also directly add the operator to the authorised list with the master station 100 by programming the operator's tag 30, in which case, the authoriser may not be required to swipe their operator tag 30.

Once the operator is added to the authorised list, they can perform the processes associated with process modules 220 and operate the cleaning unit associated with the cleaning module 240 (depending on their authorisation level).

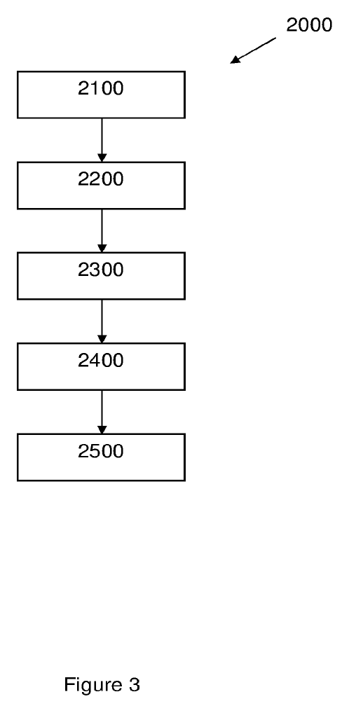

At step 2000, the operator performs a sequence of processes on the scope 10. The processes performed on the scope 10 are outlined further in FIG. 3.

At step 2100, the operator proceeds to manually wash/wipe the scope 10 down after being used on a patient. After washing/wiping down the scope 10, the operator places their operator tag 30 proximate to the first process module 220a. Following this, the operator places the tag 20 proximate to the first process module 220a. This registers on the tag 20 that the operator has initially washed/wiped down the scope 10 after being used on a patient. The identity of the operator who washed/wiped down the scope 10 is also registered on the tag 20.

Furthermore, depending on how long the operator holds the tag 20 proximate to the first process module 220a, an associated condition of the scope 10 is registered on the tag 20. In the embodiment, the operator holds the tag 20 proximate to the first process module 220a for less than a predetermined time (i.e. 4 seconds) indicating that the scope 10 is not heavily soiled. If, however, the operator held the tag 20 proximate to the first process module 220a for greater than the predetermined time, this would indicate that the scope 10 is heavily soiled and needs to be processed with a predefined time (i.e. one hour).

At step 2200, the operator proceeds to the first sink to brush the inner channels of the scope 10. After brushing the channels of the scope 10, the operator places their operator tag 30 proximate to the second process module 220b. Following this, the operator places the tag 20 proximate to the second process module 220b. This registers on the tag 20 that the operator has brushed the channels of the scope 10. The identity of the operator who brushed the channels of the scope 10 is also registered on the tag 20.

Further to the above, it would also be appreciated that the operator may register an associated condition of the scope 10 at step 2200. In addition, it will also be appreciated that if the operator missed step 2100, they will be directed back to step 2100 at step 2200 by the second process module 220b.

At step 2300, the operator proceeds to the second sink to manually wash and rinse the scope 10. After manually washing and rinsing the scope 10, the operator places their operator tag 30 proximate to the third process module 220c. Following this, the operator places the tag 20 proximate to the third process module 220c. This registers on the tag 20 that the operator has manually washed and rinsed the scope 10. The identity of the operator who manually washed and rinsed the scope 10 is also registered on the tag 20.

Further to the above, it would also be appreciated that the operator may register an associated condition of the scope 10 at step 2300. In addition, it will also be appreciated that if the operator missed steps 2100 and/or 2200, they will be directed back to either step 2100 and/or 2200 at step 2300 by the third process module 220c.

At step 2400, the operator proceeds to the leak testing apparatus to perform a leak test on the scope 10. After performing the leak test on the scope 10, the operator places their operator tag 30 proximate to the fourth process module 220d. Following this, the operator places the tag 20 proximate to the fourth process module 220d for a period of time less than the predetermined time. This registers on the tag 20 that the operator has successfully performed a leak test on the scope 10. The identity of the operator who performed the leak test on the scope 10 is also registered on the tag 20.

Further to the above, it would also be appreciated that the operator may register that the scope 10 failed the leak test and requires maintenance by, for example, placing the tag 20 proximate to the fourth process module 220d for a period of time greater than the predetermined time. In addition, it will also be appreciated that if the operator missed steps 2100, 2200 and/or 2300, they will be directed back to either step 2100, 2200 and/or 2300 at step 2400 by the fourth process module 220d.

At step 2500, the operator proceeds to perform a manual inspection on the scope 10. After manually inspecting the scope 10, the operator places their operator tag 30 proximate to the fifth process module 220e. Following this, the operator places the tag 20 proximate to the fifth process module 220e for a period of time less than the predetermined time. This registers on the tag 20 that the operator has inspected the scope 10 and it appears fit for use. The identity of the operator who performed the manual inspection on the scope 10 is also registered on the tag 20.

Further to the above, it would also be appreciated that the operator may register that the scope 10 is currently not fit for use by, for example, placing the tag 20 proximate to the fifth process module 220e for a period of time greater than the predetermined time. In addition, it will also be appreciated that if the operator missed steps 2100, 2200, 2300 and/or 2400, they will be directed back to either step 2100, 2200, 2300 and/or 2400 at step 2500 by the fifth process module 220e.

At step 3000, the scope 10 is cleaned by being put through a wash cycle of the automatic cleaning unit. It would be appreciated that if the operator has missed any one of steps 2100 to 2500, the cleaning module 240 will direct the operator back to any one of these steps when the tag is presented thereto.

A clean cycle is pre-programmed by the operator before the scope 10 is cleaned in the cleaning unit. After the scope 10 is cleaned, the operator presents their operator tag 30 to the cleaning module 240. The cleaning module 240 then records who performed the clean on the scope 10.

Following the above, the cleaning module 240 will then prompt the washer operator to present the tag 20 of the scope 10. The cleaning module 240 writes the time the scope 10 is removed as well as the washer operator detail to the tag 20. Moreover, when the tag 20 is presented to the cleaning module 240, an initial time until re-cleaning of the scope 10 will be set. This initial time will be displayed in green on the display of the cleaning module 240. Furthermore, the initial re-cleaning time of the scope 10 will be recorded by the cleaning module 240 and on the tag 20. As mentioned above, this initial time re-cleaning time will be determined by the type of clean the scope 10 has been put through (i.e. short, long, alcohol, non-alcohol etc) and the pre-programmed scope time limit parameters.

In addition, in the current embodiment, when the tag 20 is presented to the cleaning module 240, it is assumed that the scope 10 is out of the clean storage cabinet as the tag is not in communication with the status device 300. Accordingly, the time scope 10 is out of the clean storage environment is recorded.

At step 4000, the tag 20 of the scope 10 is located proximate to the status device 300 in the clean storage cabinet. That is, in the clean storage cabinet, the scope 10 is stored such that the tag 20 and status device 300 are within a short range and able to communicate. This short range prevents communication interference from other scopes 10 and status devices 300 within the clean storage cabinet. Furthermore, the storage of the scope 10 is such that the status device 300 can determine whether the scope 10 is in the clean storage cabinet. It will be appreciated that multiple status devices 300, each with a unique ID, in one or more cabinets determines where each scope tag 20 is exactly located within the system and, via the network 700, may be displayed on a screen. In addition, with information received from the tag 20, the status device 300 determines the re-cleaning time of the scope 10 according to how long the scope 10 was out of the clean storage environment and information relating to the cleaning of scope 10.

A display of the status device 300 shows the time remaining until the scope 10 should be re-cleaned. Before the tag 20 is located proximate, the status device 300 displays that no scope is present. When the tag 20 is proximate to the status device 300 and communication is established, the status device 300 will display the status of the scope 10. In particular, if the allowed time for the scope 10 being out of a clean storage environment has been exceeded, the status device will flash “00:00” in the colour red. In some cases, the scope 10 may be transferred between clean storage environments and therefore, if the allowable storage time of the scope 10 has been exceeded, the status device 300 will also flash “00:00” in the colour red. If the scope 10 has time remaining before the scope has to be re-cleaned, the status device 300 will display the time remaining until the next clean of the scope 10 in the colour green.

In certain circumstance, during the storage of the scope 10, the storage controller 400 may also communicate with the status device 300 and/or the tag 20 to adjust the remaining time until the scope 10 has to be re-cleaned. For example, if the door of the clean storage cabinet is left open, the storage controller 400 may send information to adjust the out of clean storage environment time left for the scope 10. Similarly, if a high temperature is recorded in the clean storage cabinet, the remaining storage time may be reduced. Separately, if cross-contamination is detected by, for example, a contaminated scope entering into the clean storage environment, the status device 300 may indicate that the scope 10 is now contaminated, via its display, and record that the scope 10 is contaminated on the tag 20.

If during storage, the maximum storage time of the scope 10 is exceeded, the display of the status device 300 will turn red and show the exceeded storage time of the scope 10. In this case, an operator should remove the scope 10 and take it to be re-cleaned per step 8000.

On the other hand, if the scope 10 does not need to be re-cleaned, at step S000, the scope 10 is removed from the clean storage cabinet for use during a medical operation. It would also be appreciated that the scope may proceed directly from the washer unit to be used in a medical operation.

When the scope 10 is removed from the clean storage cabinet, communication is lost between the tag 20 and the status device 300. Accordingly, this indicates that the scope is out of the clean storage environment and the time the scope 10 is out of a clean storage environment continues to be recorded. On tag 20, the remaining allowable clean storage time left for the scope 10 is also recorded.

During step 5000, a receipt from the receipt printer is collected. This receipt contains information received from the cleaning module 240 and status device 300 during steps 1000 to 4000. In particular, this receipt contains information regarding when the device was stored into the clean storage environment, when the device was removed from clean storage environment and when the scope should be re-cleaned if it is no longer stored in the cleaning storage environment.

At step 6000, the print-out from the receipt printer 600 is attached to the scope 10 and then the scope 10 is then taken off to be used in a medical operation at step 7000. Before the operation occurs, the receipt from the receipt printer 600 can be checked to ensure that scope 10 is fit for use (i.e. the scope 10 does not have to be re-cleaned as the out of clean storage time has not been exceeded). Furthermore, if one is unsure whether the scope 10 should be used, it may be returned to a reader such as the receipt printer 600 to check whether the scope 10 is still fit for use. After the scope 10 has been used in the medical procedure, it is then typically returned to the cleaning unit for re-cleaning.

As the scope 10 proceeds through steps 1000 to 8000, the system monitor 500 can be used to query the current status of the scope 10. It may also provide a summary of the recent or current activity at the cleaning unit via the cleaning module 240 and the history of the clean storage cabinet via the storage controller 400.

The device management system 1 provides facilities to allow tracking and auditing of both scope 10 and operator activities. In particular, the device management system 1 tracks the time scope 10 is in and out of a clean storage environment and when the scope should be re-cleaned. Furthermore, the process modules 220 assist in making the operator follow a sequence of maintenance/cleaning steps before the scope 10 is cleaned in the cleaning unit. This substantially assists in infection control. Similarly, the type of cleaning performed on the scope 10 is recorded, which assists in determining when the scope should be re-cleaned. Furthermore, the device management system 1 ensures that only trained operators are using the device management system 1.

Other recordable parameters may also be recorded including, but not limited to, environmental inputs, air pressure and vacuum flow either within the cabinet or within each scope 10.

In addition, the device management system 1 substantially assists in creating a maintenance schedule or cleaning schedule for the scopes 10. That is, as the device management system 1 is adapted to track multiple scopes 10 across a number of storage cabinets, a user can view the status of the scope 10 in real time and schedule which scopes 10 needs to be cleaned. Similarly, the device management system 1 assists in determining when there is a problem in the clean storage cabinet via the storage controller 400, which may require maintenance or some further attention. Furthermore, the system 1 assist in preventing cross-contamination between scopes.

The receipt printer 600 produces a duplicate copy of the storage time and shows the last time the scope can be used before re-cleaning. This avoids having to install multiple readers through out the hospital to check the status of scope 10 before use. Accordingly, the duplicate copy validates the scope as ‘fit for use’ at the bedside without additional devices or displays needed in places such as hospital theatres.

The device management system 1 is also a stand alone modular system but can interact with other networks if required. For example, the device management system 1 may be integrated with a hospital server and provide remote access. In addition, as would be appreciated by a person skilled in the art, as the tag 20 is able to store sufficient data, the process modules 220, cleaning modules 240 and/or status device 300 may be standalone units. This reduces the need for network infrastructure, as data is carried on the tag 20, and allows the management system 1 to be easily/cost effectively installed.

All devices in the device management system 1 can be re-programmed or updated externally. Use of common processors allows several devices to be re-programmed. For example, a tag reader can become a tag writer or storage controller 400, depending upon software.

The system can also be adapted for other purposes such as, but not limited to, instrument storage cabinets, asset tracking in hospitals and so forth.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step etc.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

1. A device management system, the system comprising
a tag associated with a device;
a cleaning module configured to receive a validity indication that the device has been cleaned and to record information on the tag regarding the cleaning of the device;
a status device configured to determine at least one location of the device via its proximity to the tag; and
wherein a re-cleaning time of the device is determined according to the at least one location of the device and the validity indication that the device has been cleaned.

2. The system of claim 1, wherein the cleaning module is configured to record information on the tag regarding when the device was cleaned and/or the cleaning performed on the device.

3. The system of claim 1, wherein the cleaning module is configured to record an initial time on the tag when the device should be re-cleaned.

4. The system of claim 1, wherein the status device is configured to determine the re-cleaning time of the device according to the at least one location of the device and the validity indication that the device has been cleaned.

5. The system of claim 4, wherein the status device is located in a clean storage environment.

6. The system of claim 5, wherein when the device is removed from the clean storage environment, the status device records when the device should be re-cleaned on the tag.

7. The system of claim 1, wherein the device management system includes at least one process module that allows an operator to register on the tag whether at least one process has been performed on the device.

8. The system of claim 7, wherein the at least one process includes whether the device has been washed, brushed, leak tested, rinsed and/or manually inspected.

9. The system of claim 7, wherein the at least one process module is configured to register on the tag an associated condition of the device depending on an operator input.

10. The system of claim 1, wherein the system includes a plurality of process modules that assist in making an operator follow a sequence of processes.

11. The system of claim 10, wherein in response to the operator missing a process in the sequence of processes, an indication is given to the operator by at least one of the plurality of process modules to return to a previous process.

12. The system of claim 1, wherein the device management system includes a storage controller that monitors conditions of a clean storage environment.

13. The system of claim 12, wherein depending on the conditions in the clean storage environment, the storage controller sends information to adjust the re-cleaning time until the device should be re-cleaned.

14. The system of claim 1, wherein the re-cleaning time of the device is adjusted depending on whether device is in or out of a clean storage environment.

15. The system of claim 1, wherein the status device is configured to determine cross-contamination by determining whether the validity indication that the device has been cleaned was received.

16. The system of claim 9, wherein the operator input includes swiping the tag proximate to the at least one process module above a predetermined time to register the associated condition of the device.

17. The system of claim 16, wherein swiping the tag proximate to the at least one process module above the predetermined time indicates that the device is heavily soiled.

18. The system of claim 9, wherein the status device is configured to determine cross-contamination by determining whether the device has been processed correctly through the at least one process module.

19. The system of claim 1, wherein a receipt printer is configured to receive information from the tag and print a receipt that is checked to ensure that the device is fit for use prior to an operation.

20. A method of determining a cleaning cycle of a device, comprising:
receiving an indication that the device has been cleaned on a tag associated with the device;
determining at least one location of the device via the tag, after receiving the indication that the device has been cleaned; and
determining a re-cleaning time of the device according to the at least one location and the indication that the device has been cleaned.

21. The method of claim 20, wherein the step of receiving the indication that the device has been cleaned includes receiving an indication from a cleaning unit at a cleaning module.

22. The method of claim 20, wherein before receiving an indication that the device has been cleaned, the method further includes a step of adding an operator to an authorized list.

23. The method of claim 20, wherein the step of determining the at least one location of the device via the tag includes determining whether the device is in a clean storage environment.

24. The method of claim 20, wherein before receiving an indication that the device has been cleaned, the method further includes a step of registering whether at least one process has been performed with the device.

25. The method of claim 24, wherein the step of registering whether the at least one process has been performed with the device includes registering an associated condition of the device.

26. The method of claim 20, wherein the step of determining the re-cleaning time of the device includes adjusting the re-cleaning time of the device depending on whether the device is in or out of a clean storage environment.

27. The method of claim 24, wherein the at least one process includes whether the device has been washed, brushed, leak tested, rinsed and/or manually inspected.

28. The method of claim 25, wherein the step of registering the associated condition of the device includes swiping the tag proximate to at least one process module above a predetermined time.

29. The method of claim 28, wherein swiping the tag proximate to the at least one process module above the predetermined time indicates that the device is heavily soiled.

30. The method of claim 20, wherein the method further includes determining whether the device has been cross contaminated from one or more other devices.

31. The method of claim 20, wherein the method further includes printing a receipt that is checked to ensure that the device is fit for use prior to an operation.
